(19) United States
(12) Patent Application Publication
(10) Pub. No.: US 2020/0187576 A1
(43) Pub. Date: Jun. 18, 2020

(54) INNER BITE RING

(71) Applicant: Ayako IZUMI, Tokyo (JP)

(72) Inventor: Ayako IZUMI, Tokyo (JP)

(21) Appl. No.: 16/621,213

(22) PCT Filed: Jun. 13, 2018
(86) PCT No.: PCT/JP2018/022528

§ 371 Date: Dec. 10, 2019

(30) Foreign Application Priority Data
Jun. 30, 2017 (JP) 20170128447

(51) Int. Cl. A41D 13/08; A61B 42/20; A61C 5/00

(52) U.S. Cl. CPC A41D 13/087; A61C 5/007; A61B 42/20

(57) ABSTRACT

Provided is an inner bite ring which can be used by wearing a glove on a finger wearing the inner bite ring, is unlikely to damage the glove, and does not damage tissue around an oral cavity or teeth even if it is bitten from over the glove, for maintaining an opening and guarding the finger. An inner bite ring 1 has: a tubular portion 2 into which a finger of a human hand is inserted, the glove being worn on the inner bite ring 1 worn on the finger; a soft elastic portion 3 coated on an outer periphery of the tubular portion 2; and a strap portion 4 connected to a root portion 2b side of the tubular portion 2 and hung on a wrist or an adjacent finger. The tubular portion 2 is closed at its tip, and has a vent hole 6 formed on a tip portion side to communicate an inside and an outside of the tubular portion 2.

### TECHNICAL FIELD

The present invention relates to a technique for maintaining an opening and guarding a finger during an oral care treatment or the like.

### BACKGROUND ART

An instrument (a mouth gag) for maintaining the opening and guarding the finger during oral care, treatment, or the like has been proposed (PATENT LITERATURE 1). The mouth gag has a configuration in which a tubular cushion material formed in a cylindrical shape with a soft synthetic resin material is provided in an outer tube formed in a cylindrical shape with a hard synthetic resin material.

With the mouth gag, a practitioner performs oral care and treatment in a state in which the practitioner inserts the finger into the tubular cushion material, for example, at a medical or nursing care site. At that time, even if the outer tube inserted in an oral cavity is bitten by teeth, the finger of the practitioner is not damaged and tissue around the oral cavity is not easily damaged. Moreover, the tubular cushion material fits the inserted finger. The mouth gag can also be used together with a typical instrument.

### CITATION LIST

### Patent Literature

PATENT LITERATURE 1: Japanese Utility Model No. 3125738

### SUMMARY OF THE INVENTION

### Problems to Be Solved By the Invention

Currently, when performing oral care, treatment, or the like, the practitioner often wears the mouth gag on the finger from over a glove with the glove worn on a hand in advance; however, it is necessary to disinfect the mouth gag after each operation. Therefore, it is desirable to wear the glove on the hand in a state in which the instrument for maintaining the opening of the mouth gag or the like and guarding the finger is previously worn on the finger.

However, when wearing the glove, which is formed of latex or the like that fits the hand, and is in close contact with the hand, from over the typical mouth gag worn on the finger, since the outer tube is formed of the hard synthetic resin material, biting teeth hit the outer tube and easily damage the glove. Further, if an end or the like of the outer tube is sharp, the glove may be damaged when the glove is worn on the outer tube.

An object of the present invention is to provide an inner bite ring which can be used by wearing the glove on the finger wearing the inner bite ring, is unlikely to damage the glove, and does not damage the tissue around the oral cavity or the teeth even if it is bitten from over the glove, for maintaining the opening and guarding the finger.

### Solution to the Problems

### [Invention 1]

In order to achieve the above object, the inner bite ring of Invention 1 is an inner bite ring which is worn on a finger of a human hand and on which a glove is worn, for maintaining an opening and protecting the finger from bite, including: a tubular portion into which the finger is inserted; and a soft elastic portion coated on an outer periphery of the tubular portion.

### [Invention 2]

The inner bite ring of Invention 2 is the inner bite ring according to Invention 1, wherein the tubular portion is closed at its tip, and has a vent hole formed on a tip portion side to communicate an inside and an outside of the tubular portion.

### [Invention 3]

The inner bite ring of Invention 3 is the inner bite ring according to Invention 1 or 2, wherein a strap portion is connected to a root portion side of the tubular portion.

### [Invention 4]

The inner bite ring of Invention 4 is the inner bite ring according to any one of Inventions 1 to 3, wherein a surface of the soft elastic portion is formed as an uneven surface.

### [Invention 5]

The inner bite ring of Invention 5 is the inner bite ring according to any one of Inventions 1 to 4, wherein an outer peripheral edge portion of the soft elastic portion is formed as an inclined surface.

### [Invention 6]

The inner bite ring of Invention 6 is the inner bite ring according to any one of Inventions 1 to 5, wherein the finger wearing the inner bite ring can be bent due to material or form.

### Effects of the Invention

As described above, according to the inner bite ring of Invention 1, the glove can be exchanged while the inner bite ring is worn on the finger, and a need for sterilization and disinfection of the inner bite ring in each time is reduced. Further, even if a bite force is strong, the finger is not damaged, and the tissue around the oral cavity and the teeth are not damaged due to the elastic portion. Further, since the soft elastic portion is soft, the glove is unlikely to be cut by the biting teeth, and safety is increased. Further, since the inner bite ring is worn on the practitioner's own finger, it is more stable than the instrument, and is unlikely to come off unless the practitioner removes it for some reason. Furthermore, since it is worn under the glove, there is less risk of breakage or accidental ingestion.

According to the inner bite ring of Invention 2, when inserting the finger into the inner bite ring, air on the tip side is discharged out of the inner bite ring through the vent hole, so that the inner bite ring can be worn smoothly.

According to the inner bite ring of Invention 3, the inner bite ring can be prevented from falling into the glove when the glove is removed by hanging the strap portion on a wrist, an adjacent finger, or the like.

According to the inner bite ring of Invention 4, a frictional force between the glove and the surface of the soft elastic portion can be suppressed low, and the glove can be easily worn or removed.

According to the inner bite ring of Invention 5, frictional resistance of the soft elastic portion against the glove can be reduced, and the glove can be smoothly worn or removed.

According to the inner bite ring of Invention 6, since the finger wearing the inner bite ring can be bent, palpation is easy.

### BRIEF DESCRIPTION OF THE DRAWINGS

FIG. 1 is a partially broken external perspective view showing a first embodiment of an inner bite ring according to the present invention.

FIG. 2 is a view showing a state in which a glove is worn on an index finger wearing the inner bite ring shown in FIG. 1.

FIG. 3 is a plan view seen from a back of a hand showing the state in which the glove is worn on the index finger wearing the inner bite ring shown in FIG. 1.

FIG. 4 is a partially broken external perspective view showing a second embodiment of the inner bite ring according to the present invention.

FIG. 5 is a view showing a state in which the glove is worn on the index finger wearing the inner bite ring shown in FIG. 4.

### DESCRIPTION OF THE EMBODIMENTS

Hereinafter, the present invention will be described in detail based on embodiments shown in the drawings.

### First Embodiment

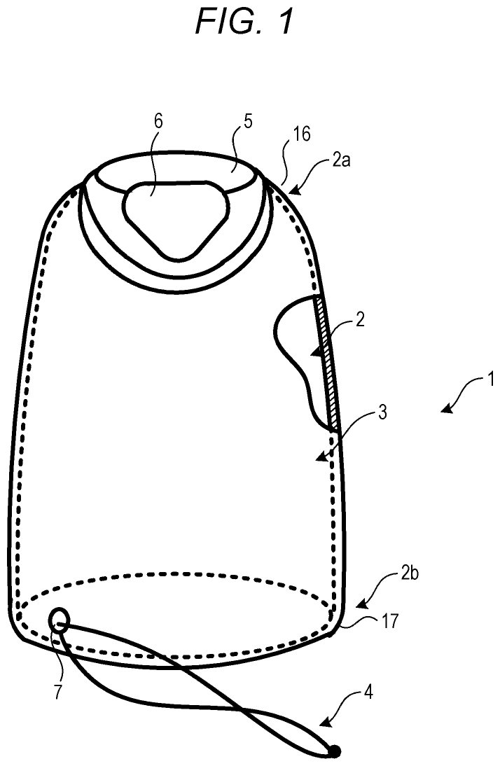

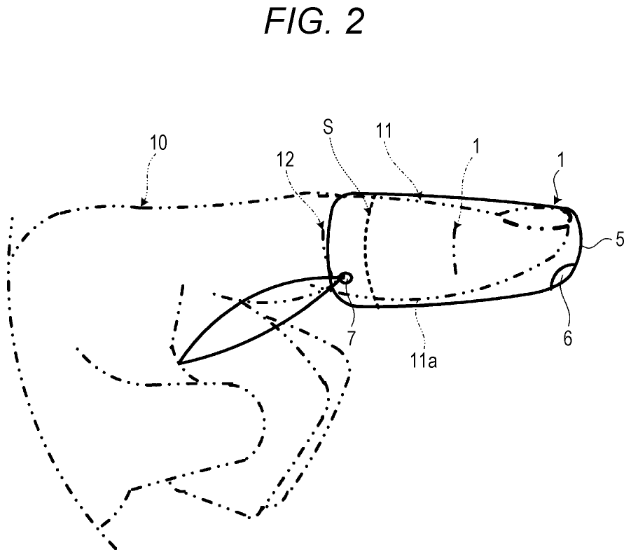

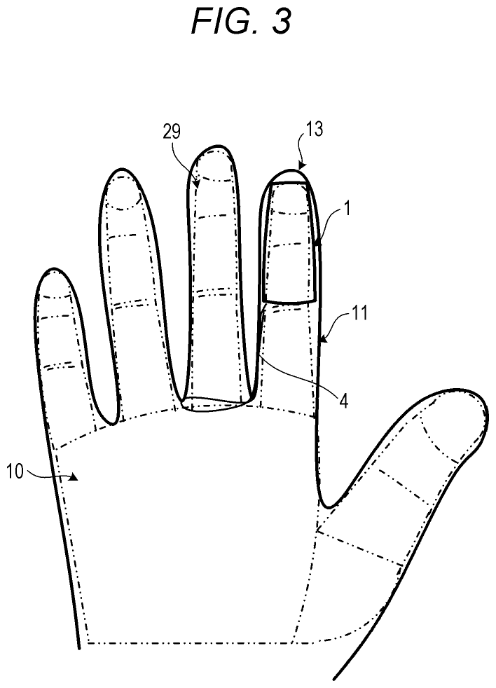

FIG. 1 is a partially broken external perspective view showing a first embodiment of an inner bite ring according to the present invention. FIG. 2 is a view showing a state in which a glove is worn on an index finger wearing the inner bite ring shown in FIG. 1. FIG. 3 is a plan view seen from a back of a hand showing the state in which the glove is worn on the index finger wearing the inner bite ring shown in FIG. 1.

An inner bite ring 1 includes a tubular portion 2 formed in a cylindrical shape with a metal or a hard synthetic resin material, a soft elastic portion 3 made of a soft elastic member such as urethane rubber coated on an outer peripheral surface of the tubular portion 2, and a strap portion 4 formed in a ring shape with a rubber string or the like.

The tubular portion 2 has a tip portion 2a closed by a flat top portion 5, and a root portion 2b opened. The tubular portion 2 is formed in a tapered shape having an outer diameter gradually reduced from the root portion 2b side toward the tip portion 2a. Note that an outer shape of the tubular portion 2 may be a cylindrical shape having a constant outer diameter.

The tubular portion 2 has a vent hole 6 formed in an outer peripheral portion of the top portion 5. As shown in FIG. 2, the inner bite ring 1 has the tubular portion 2 worn on a fingertip of an index finger 11 of a practitioner's hand (for example, a doctor, a dentist, a nurse, or the like) 10. When the fingertip is inserted into the tubular portion 2, air in the tip portion of the tubular portion 2 is discharged from the vent hole 6 to an outside of a tube, so that the tubular portion 2 can be inserted up to the fingertip. A position where the finger is inserted into the tubular portion 2 is not particularly defined; however, as shown in FIG. 2, if the tubular portion 2 is worn with the vent hole 6 aligned with a ball 11a of the index finger 11, the air can be easily discharged.

The tubular portion 2 is set to a length that allows the root portion 2b to be inserted up to a proximal interphalangeal joint (PIP joint) 12. Further, an inner diameter of the tubular portion 2 is desirably such that it can be lightly pushed into the finger to be worn. Therefore, it is preferable to prepare a plurality of sizes of inner bite rings 1 having different inner diameters and lengths. The inner diameter of the tubular portion 2 can be exemplified by about 17 mm to 20 mm in consideration of the outer diameter of the PIP joint 12, but is not limited thereto.

In addition, as shown in FIG. 2, a ring-shaped spacer S formed of rubber or the like is bonded to an inner peripheral surface of the tubular portion 2 on the root portion 2b side, so that the index finger 11 can be held in the tubular portion 2 without slip even if a finger size does not match the tubular portion 2.

A through-hole 7 of the strap portion 4 is formed on the root portion 2b side of the tubular portion 2, and the rubber string of the strap portion 4 is inserted into and connected to the through-hole 7. The strap portion 4 is wound around, for example, a wrist or a middle finger, and prevents the inner bite ring 1 from falling off the index finger 11 wearing the inner bite ring 1.

The soft elastic portion 3 is made of a material softer than the teeth, and for example, a urethane rubber sheet is bonded to the outer peripheral surface of the tubular portion 2 with an adhesive, a double-sided adhesive tape, or the like. Therefore, the soft elastic portion 3 can be exchanged. Of course, as a method of coating an outer periphery of the tubular portion 2 with the soft elastic portion 3, a spray method, a dipping method, or the like may be employed. A coating thickness of the soft elastic portion 3 can be exemplified by about 1 mm, but is not limited thereto. It is only necessary to have a thickness that can reduce the bite force and a thickness that does not damage the oral cavity.

In the present embodiment, as shown in FIG. 3, a glove 13 is worn on the hand 10 with the inner bite ring 1 being worn on the index finger 11 in advance. In this case, if a frictional force between the glove 13 and the soft elastic portion 3 is high, there is a possibility that the glove 13 cannot be worn smoothly. Therefore, by making a surface of the soft elastic portion 3 a rough surface with fine irregularities formed like a satin surface, the surface of the soft elastic portion 3 is prevented from being in close contact with an inner surface of the glove 13, and the glove 13 can be smoothly worn from over the inner bite ring 1. Since the urethane rubber sheet has numerous fine holes formed on the surface, the glove 13 can be smoothly worn from over the inner bite ring 1.

Moreover, the soft elastic portion 3 allows the vent hole 6 to vent the air by exposing the vent hole 6 through a vent hole cutout portion 14 cut and formed around the vent hole 6. Further, the rubber string of the strap portion 4 can be inserted into the through-hole 7 through an opening portion 15 cut and formed around the through-hole 7.

The soft elastic portion 3 is coated on the outer periphery of the tubular portion 2 except for the top portion 5 of the tubular portion 2 where there is no possibility of biting and an opening of the root portion 2b. An inclined surface is formed in which a thickness of an outer periphery 16 of the tip portion 2a gradually decreases toward a tip end thereof, and frictional resistance when wearing the glove 13 on the inner bite ring 1 is made as small as possible. Further, an inclined surface is formed in which a thickness of an outer periphery 17 of the root portion 2b gradually decreases toward a rear end, and the frictional resistance when removing the glove 13 is made as small as possible. Therefore, when removing the glove 13, the inner bite ring 1 is prevented from being dragged by the glove 13 and left in the glove 13. That is, an outer peripheral edge portion of the soft elastic portion 3 is formed as the inclined surface.

As shown in FIG. 3, the inner bite ring 1 according to the present embodiment is worn, for example, on a tip of the index finger 11 of the hand 10 in advance by the doctor, the dentist, a care worker, or the like, and the glove 13 is worn on the hand 10 with the strap portion 4 being hung on another finger or the like. At that time, since the surface of the soft elastic portion 3 has low friction with respect to the glove 13, the glove 13 can be smoothly worn on the hand.

Since the inner bite ring 1 is covered with, for example, a translucent glove 13, wearing of the inner bite ring 1 is not visible by a patient or the like, and the patient or the like can relax, open mouth, and receive treatment.

Further, the inner bite ring 1 can be used by caregivers and parents at home. It can also be used for pets in some cases.

When the inner bite ring 1 is applied to the teeth to maintain the opening, even if the bite force becomes strong, and the teeth bite into the inner bite ring 1 through the glove 13, the finger of the practitioner is protected by the tubular portion 2 and the teeth are not damaged due to soft elasticity of the soft elastic portion 3.

Further, since the soft elastic portion 3 has soft elasticity, it does not function as a cutting board by biting with the teeth, and thus the glove 13 can be prevented from being torn.

Therefore, the practitioner need only replace the glove 13 at the time of subsequent oral care, treatment, or the like, and can save time and effort to sterilize and disinfect the inner bite ring 1. In particular, when medical environment is in a bad state, such as when a disaster occurs, the practitioner can perform the subsequent treatment by simply replacing the glove 13 while wearing the inner bite ring 1 on the finger.

### Second Embodiment

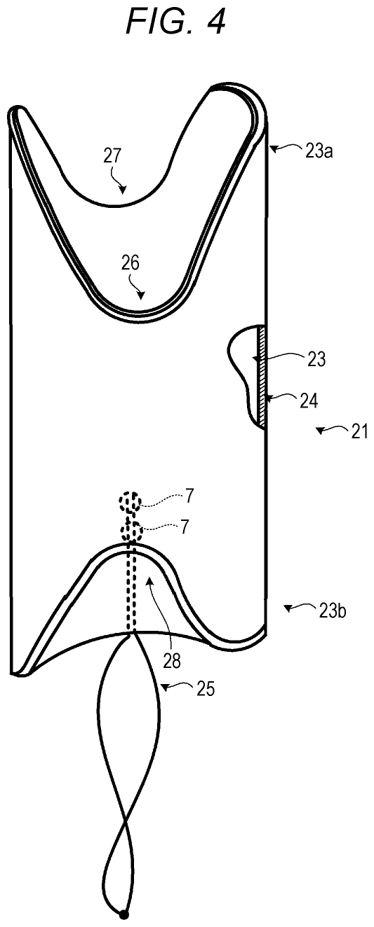

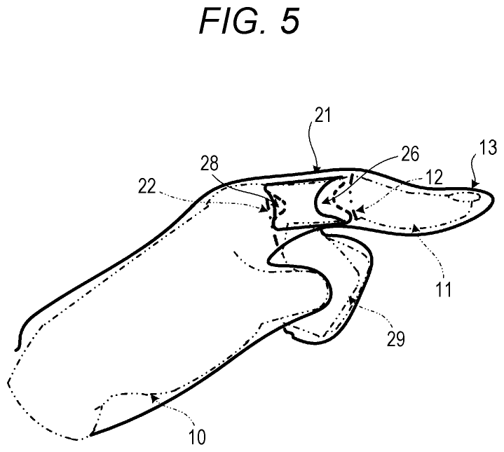

FIG. 4 is a partially broken external perspective view showing a second embodiment of the inner bite ring according to the present invention. FIG. 5 is a view showing a state in which the glove is worn on the index finger wearing the inner bite ring shown in FIG. 4.

An inner bite ring 21 of the present embodiment is used by being inserted up to a base of the index finger 11 and is worn between a metacarpophalangeal joint (MP joint) 22 and the PIP joint 12 as shown in FIG. 5. Since a strap portion 25 is the same as the strap portion 4 of the first embodiment, description thereof will be omitted.

The inner bite ring 21 includes a tubular portion 23 formed in a cylindrical shape with a metal or a hard synthetic resin material, a soft elastic portion 24 made of a soft elastic member such as urethane rubber coated on the outer peripheral surface of the tubular portion 23, and the strap portion 25 formed in a ring shape with the rubber string or the like.

The tubular portion 23 is formed in a cylindrical shape having a constant inner diameter, and an upper first cutout portion 26 and an upper second cutout portion 27 that are cut into a curved shape with the tip end open are formed facing each other at a tip portion 23a. A lower cutout portion 28 cut into a curved shape with the root open is formed at a root portion 23b. The upper first cutout portion 26 and the lower cutout portion 28 are formed on the same side surface so as to be vertically separated from each other. Although a cutout portion having the same shape as the lower cutout portion 28 is not provided in a facing surface of the lower cutout portion 28, it may be provided.

As shown in FIG. 5, the inner bite ring 21 is worn such that the upper first cutout portion 26 and the upper second cutout portion 27 of the tubular portion 23 are positioned on the side surface side of the index finger 11. Further, the lower cutout portion 28 is placed in a crotch between the index finger 11 and a middle finger 29. The upper first cutout portion 26, the upper second cutout portion 27, and the lower cutout portion 28 ensure free movement of the index finger 11 without hindering the movement of the MP joint 22 and the PIP joint 12.

The soft elastic portion 24 is configured by removably bonding the urethane rubber sheet to the outer peripheral surface of the tubular portion 23 in the same manner as the soft elastic portion 3 of the first embodiment. The soft elastic portion 24 has the urethane rubber sheet cut out along opening edges of the tip portion 23a and the root portion 23b of the tubular portion 23. A cutout edge portion of the urethane rubber sheet is formed as the inclined surface, and when the glove 13 is worn or removed, it does not hinder smooth wearing and removal of the glove 13 as in the first embodiment.

The strap portion 25 is made of the rubber string connected through two through-holes 7 formed in the tubular portion 23 and opening portions (not shown) of the soft elastic portion 24 formed around the through-holes 7.

According to the inner bite ring 21 of the present embodiment, the same effect as that of the first embodiment is obtained. In addition, when the practitioner performs treatment or the like while the finger is inserted deeply into the oral cavity so that the MP joint 22 and the PIP joint 12 of the index finger 11 touch the teeth, it is possible to protect the index finger 11 from being bitten. Further, since the fingertip is out of the inner bite ring 21, the palpation can be performed in some cases. In particular, as in the present embodiment, the palpation is easy if it is configured so that the worn finger can be bent due to the material or form.

### LIST OF REFERENCE NUMERALS

1, 21 Inner bite ring
2, 23 Tubular portion
2a, 23a Tip portion
2b, 23b Root portion
3, 24 Soft elastic portion
4, 25 Strap portion
5 Top portion
6 Vent hole
7 Through-hole
10 Practitioner's hand
11 Index finger
11a Ball
12 Proximal interphalangeal joint (PIP joint)
13 Glove
14 Vent hole cutout portion
15 Opening portion
16, 17 Outer periphery
22 Metacarpophalangeal joint (MP joint)
26 Upper first cutout portion
27 Upper second cutout portion
28 Lower cutout portion
29 Middle finger
S Spacer

1. An inner bite ring which is worn on a finger of a human hand and on which a glove is worn for maintaining an opening and protecting the finger from bite, the inner bite ring comprising:
a hard tubular portion into which the finger is inserted; and
a soft elastic portion coated on an outer periphery of the tubular portion.

2. The inner bite ring according to claim 1, wherein the tubular portion is closed at its tip, and has a vent hole formed in an outer peripheral portion on a tip portion side to communicate an inside and an outside of the tubular portion.

3. The inner bite ring according to claim 1, wherein a strap portion is connected to a root portion side of the tubular portion.

4. The inner bite ring according to claim 1, wherein a surface of the soft elastic portion is formed as an uneven surface.

5. The inner bite ring according to claim 1, wherein an outer peripheral edge portion of the soft elastic portion is formed as an inclined surface.

6. The inner bite ring according to claim 1, wherein the finger wearing the inner bite ring can be bent due to material or form.
